(19) United States
(12) Patent Application Publication
(10) Pub. No.: US 2005/0049209 A1
(43) Pub. Date: Mar. 3, 2005

(54) Pharmaceutical compositions for delivering macrolides

(76) Inventor: Andrew Xian Chen, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/895,018

(22) Filed: Jul. 20, 2004

Related U.S. Application Data
(60) Provisional application No. 60/493,209, filed on Aug. 6, 2003.

(51) Int. Cl. A61K 31/7048; A61K 31/4745; A61K 35/78

(52) U.S. Cl. 514/28; 514/291; 424/776

(57) ABSTRACT

The present invention provides injectable macrolide oil-in-water emulsions and lyophilized formulations thereof. The present invention also provides methods for preparing and using such oil-in-water emulsions and lyophilized formulations thereof.

### CROSS-REFERENCE TO RELATED APPLICATION

This application claims the benefit of U.S. Provisional Patent Application No. 60/493,209, filed Aug. 6, 2003, where this provisional application is incorporated herein by reference in its entity.

### BACKGROUND OF THE INVENTION

1. Field of the Invention

The present invention relates to pharmaceutical compositions for delivering macrolides.

2. Description of the Related Art

Macrolide antibacterials possess activity against a wide range of bacterial pathogens. Erythromycin, the first macrolide that was developed, is effective against Streptococcus pneumoniae, Mycoplasma pneumoniae, legionella pneumophilia and Chlamydia trachomatis (Alvarez-Elcoro et al., The macrolides: erythromycin, clarithromycin, and azithromycin. Mayo Clin Proc 74: 613-34,1999). The newer macrolides—such as clarithromycin, a methoxy derivative of erythromycin—have extended spectra of activity and have proved effective against HIV-related opportunistic infections, such as mycobacterium avium complex diseases (Kissinger et al., Comparison of multiple drug therapy regiments for HIV-related disseminated mycobacterium avium complex disease. J Acquir Immune Defic Syndr Hum Retrovirol 9: 133-7,1995).

For certain patients who cannot take oral medications, or who may have severe infections, initial intravenous treatment may be necessary. An intravenous formulation of erythromycin has been available and in clinical use for many years. However, its usefulness can be limited by a high incidence of adverse gastrointestinal (GI) effects (Kapusnik-Kner et al., The pharmacological basis of therapeutics. 9^{th} ed. New York: McGrawHill, p1135-40, 1996). In addition, phlebitis can occur when intravenous erythromycin is administered at a recommended concentration, i.e. 1-2 mg/mL (Caforio G. IV clarithromycin vs combined IV therapy with cefuroxime and erythromycin for pneumonia in hospitalized patients. Second International Conference on Macrolides, Azalides and Streptogramins: 19-22 Jan. 1994; Venice, p.65).

An alternative intravenous macrolide therapy has been clarithromycin. In addition to its broader antibiotic spectrum, clarithromycin also reportedly relates to a lower incidence and less severe adverse gastrointestinal (GI) effects compared to erythromycin. However, the application of intravenous clarithromycin is relatively limited: The formulation (Klaricid® by Abbott Labs) is approved only in the United Kingdom and certain other European countries, and is not licensed in the United States. It has been indicated that the local tolerability of intravenous clarithromycin is very problematic and is no better than that of erythromycin (Torsten Zimmerman et al., Comparative tolerability of intravenous azithromycin, clarithromycin and erythromycin in healthy volunteers: results of a double-blind, double-dummy, four-way crossover study. Clinical Drug Investigation 21: 527-36, 2001). For example, Zimmerman et al. reported that, with a high incidence and severity, adverse events at the injection sites from the intravenous clarithromycin (Klaricid®) administration were phlebitis (50%), vein inflammation (75%) and vein irritation (100%).

In general, macrolides, such as erythromycin and clarithromycin, belong to the class of lipophilic compounds (i.e., compounds that are water-insoluble) and are known for causing venous irritation/pain on injection. Accordingly, macrolides are generally given intravenously in dilute (2 mg/mL) solutions by slow infusion (total daily doses can be in gram quantities).

Clarithromycin freebase is substantially insoluble in water but can be solubilized at a low pH (pH<5), at which clarithromycin forms a salt. For example, clarithromycin can be converted to a lactobionate (as in the Klaricid® product) or glucoheptonate salt, and the resulting salt is soluble in water at pH 3-4. Such solution, however, displays the aforementioned venous irritation. It was postulated that the drug in a low pH salt form would again become insoluble or precipitate out in a pH neutral environment such as blood, and therefore, result in vein irritation. The relative lipophilicity of clarithrymycin has led various investigators to propose a variety of lipid dispersed systems, such as liposomes, mixed micelles, etc., which might shield the drug from contact with sensitive tissues at the injection size. To this date, however, none of these efforts has advanced as far as clinical development.

PCT Publication No. WO9014094 (Hui et al., 1990) describes injectable clarithromycin oil-in-water (o/w) fat emulsion compositions, which are comprised of triglycerides (such as soybean oil) as the lipid phase, egg lecithin as the emulsifier, oleic/hexanoic acids as the stabilizer, and glycerin as the tonicity agent. The PCT publication discloses the use of a stabilizer, for example, the combination of oleic/hexanoic acids, is required for improving clarithromycin solubility and stability in the fat emulsion. However, the use of oleic and hexanoic acids has been rare in any injection formulation marketed. In fact, the U.S. Food and Drug Administration (FDA) has not approved the application of oleic acid or hexanoic acid for its use in intravenous injection formulations (see, http://www.accessdata.fda.gov/scripts/cder/iig/index.cfm). This no-approval situation is possibly related to safety and toxicity issues of the oleic/hexanoic acids.

U.S. Pat. No. 6,479,540 B1 (Constantinides et al., 2002) discloses tocol-soluble ion pair formulation of clarithromycin for intravenous administration. This clarithrymocin formulation is an oil-in-water fat emulsion. The oil phase comprises 5% delta-tocopherol and 2.5% Capmul MCM, by weight of the final oil-in-water emulsion; the emulsifier used was poloxamer 407, 3% by weight; the ion-pair agent (used to solubilize clarithromycin by converting it to a more lipophilc compound) was vitamin E succinate, 0.9% by weight.

Again, the FDA has not approved the use of delta-tocopherol and vitamin E succinate in an intravenous injection product (see, http://www.accessdata.fda.gov/scripts/cder/iig/index.cfm). In 1983, E-Ferol, a vitamin E emulsion was introduced for vitamin E supplementation and therapy in neonates. Within a few months, more than 30 babies had died as a result of receiving the product, which resulted in prompt withdrawal of the product from the market by the FDA (Alade et al., Pediatrics 77(4): 593-597,1986). To date, various research efforts have been directed to solving some of the problems confronting vein irritation, but at the expense of leaving some equally important problems unresolved.

U.S. Pat. No. 5,958,888 (Macy et al., 1999) discloses water miscible pharmaceutical compositions containing up to about 40% of a macrolide antibiotic by reaction of the macrolide with an acid in a non-aqueous water miscible organic solvent system. One of the compositions given in the patent utilized 40% N-methyl pyrrolidone and 36% propylene glycol, by weight, as vehicle.

However, the formulation compositions disclosed by Macy et al. are of solution nature and thus fall outside of the oil-in-water fat emulsion category discussed earlier. As a result of this difference, macrolides (e.g., erythromycin or clarithromycin) as formulated in U.S. Pat. No. 5,958,888 would be expected to cause vein irritation due to the exposed contact with tissues at the injection site. In addition, the application of N-methyl pyrrolidone for intravenous injection has not been approved by regulatory agencies for safe use in humans.

U.S. Pat. No. 5,091,188 (Haynes, 1992) discloses a technique for preparing water-insoluble drugs in injectable formulations as aqueous suspensions of phospholipid-coated microcrystals. The crystalline drug is reduced to 50 nm to 10 micron dimensions by sonication or other process inducing high shear in the presence of phospholipid or other membrane-forming amphipathic lipid. The membrane-forming lipid stabilizes the microcrystal by both hydrophobic and hydrophilic interactions, coating and enveloping it and thus protecting it from coalescence, and rendering the drug substance in solid form less irritating to tissue.

Based on the invention, the coating and enveloping of the microcrystalline water-insoluble drug particles may seem to harvest the benefit of reducing the vein irritation problems associated with macrolides solution (U.S. Pat. No. 5,958,888). However, there exist a few new problems with the application of this technique. First, the size distribution (5 nm to 10 micron) for the microcrystalline drug particles is extremely wide. The result of this would be the uneven thickness of phospholipid coating around the microcrystals. A further implication of this uneven phospholipid coating is the unpredictable drug release pattern following injection from the different coating layers. For example, fast release would be the result of thinner phospholipid coating, whereas slow release would be the result of thicker phospholipid coating. In addition, because the drug particles exist in their solid form coated by phospholipids, the rate of their dissolution also remains unpredictable following injection, depending on the water-solubility of the drug and other physico-chemical parameters of the formulations. If these phospholipid-coated drug microcrystals remain insoluble in the blood stream at high concentrations, the possibility of blood vessel clogging is a significant safety issue for patients.

U.S. Pat. No. 5,085,864 (Cannon et al., 1992) discloses an intravenous injection composition containing micelles for the delivery of macrolides such as erythromycin and clarithromycin. The disclosed technique utilizes bile salt such as sodium glycodeoxycholate as the micelle formation platform. However, bile salts are known to be hemolytic agents, and have been approved by regulatory agencies for use in intravenous injection formulations only for very severe illness such as systemic fungal infection. This bile salt solubilized clarithromycin formulation is of solution in nature and thus would be expected to cause vein irritation due to the exposed contact with tissues at the injection site.

In light of these problems confronting injectable clarithromycin compositions, there exists a need for developing a vehicle that can be used for delivering lipophilic and vein-irritating compounds, such as macrolides. The present invention satisfies this need and provides other related advantages.

### BRIEF SUMMARY OF THE INVENTION

The present application provides pharmaceutical compositions for delivering macrolides and methods for making and using such compositions. The compositions of the present invention have one or more of the following properties: (1) injectable, (2) in the form of an oil-in-water emulsion, (3) stable under appropriate storage conditions, (4) vein non-irritable, (5) containing pharmaceutically effective amount of a macrolide, (6) sterilizable by filtration, (7) containing components acceptable by regulatory agencies (e.g., the FDA), and (8) not causing hyperlipodemia or other side effects.

In one aspect, the present invention provides an injectable oil-in-water emulsion that comprises (a) a pharmaceutically effective amount of a macrolide, (b) an oil component at a concentration of at most 10% by weight, (c) one or more phospholipids at a total concentration between about 1.2% to about 5% by weight, and (d) water.

In certain embodiments, the emulsion contains a macrolide at a concentration at least 0.2%, 0.3%, 0.4%, 0.5%, 0.6%, 0.7%, 0.8%, 0.9%, or 1.0% by weight.

In certain embodiments, the macrolide is clarithromycin, erythromycin, or azithromycin.

In certain embodiments, the oil component comprises a vegetable oil.

In certain embodiments, the oil component comprises a vegetable oil and a medium chain triglycerol. In certain embodiments, the weight ratio of the vegetable oil to the medium chain triglycerol is about 9:1 to about 1:1.

In certain embodiments, the emulsion further comprises a stabilizer, such as glycine and EDTA.

In certain embodiments, the emulsion further comprises a tonicity modifier, such as glycerol.

In certain embodiments, the average size of the oil droplets in the emulsion is less than about 500 nm, 400 nm, 300 nm, 200 nm, 150 nm or 100 nm.

In another aspect, the present invention provides an injectable oil-in-water emulsion that comprises (a) clarithromycin at a concentration of about 0.5% or higher by weight, (b) a medium chain triglycerol (e.g., Miglyol 812) at a concentration of about 1% to about 5% by weight, (c) a vegetable oil (e.g., soybean oil) at a concentration of about 5% to about 9% by weight, (d) a phospholipid (e.g., soy lecithin or egg lecithin) at a concentration of about 3% by weight, and (e) water.

In certain embodiments, the emulsion may further comprise glycine at a concentration of about 1%, a tonicity modifier (e.g., a glycerol) at a concentration of about 1.5%, and/or EDTA at a concentration of about 0.005%.

In another aspect, the present invention provides an injectable oil-in-water emulsion that comprises (a) a macrolide at a therapeutically effective concentration, (b) an oil component, (c) an emulsifier, and (d) water, wherein the emulsion does not cause vein irritation and is stable for at least 3 months.

In certain embodiments, the oil component comprises a vegetable oil.

In certain embodiments, the oil component comprises a vegetable oil and a medium chain triglycerol. In certain embodiments, the weight ratio of the vegetable oil and the medium chain triglycerol is about 9:1 to about 1:1.

In certain embodiments, the emulsifier is a phospholipid.

In certain embodiments, some or all of the individual components of the emulsion other than the macrolide are generally regarded as safe for use in travenous injections by a drug regulatory authority.

In another aspect, the present invention provides a lyophilized formulation of a macrolide, wherein the formulation, when hydrated, produces the oil-in-water emulsions as described herein.

In certain embodiments, the average droplet size of the rehydrated emulsion is no more than about 500%, 300%, or 150% of the average droplet size of the emulsion before the freeze-drying.

In another aspect, the present invention also provides a method for preparing an injectable oil-in-water emulsion that contains a pharmaceutically effective amount of a macrolide. The method comprises (a) forming a mixture that comprises (i) a pharmaceutically effective amount of a macrolide free base, (ii) an oil component (e.g., a vegetable oil, or a combination of a vegetable oil and a medium chain triglyceride), and (iii) a phospholipid, (b) forming an oil-in-water emulsion with the mixture of step (a) and an aqueous solution, (c) adjusting the pH of the emulsion of step (b) to about 2-5, and (d) re-adjusting the pH of the emulsion resulting from step (c) to about 6-8 to provide an injectable oil-in-water emulsion that contains a pharmaceutically effective amount of the macrolide.

In certain embodiments, step (a) may be performed by dissolving the macrolide in a solution (e.g., ethanol) and mixing the dissolved macrolide with a composition that comprises the oil component and the phospholipid.

In certain embodiments, step (b) may be performed by adding the aqueous solution to the mixture of step (a) via mechanical homogenization.

In another aspect, the present invention also provides a method of treating bacterial and/or other microbial infection by administering to a subject in need thereof a pharmaceutically effective amount of an injectable oil-in-water emulsion described herein that comprises a macrolide.

In certain embodiments, the administration may be intravenous, intramuscular, intra-arterial, intrathecal, intraocular, subcutaneous, intraarticular and intra-peritoneal.

### BRIEF DESCRIPTION OF THE DRAWINGS

FIG. 1 shows representative chromatograms of clarithromycin.

FIGS. 2A-2F show histological analysis of marginal ear vein of New Zealand white rabbits injected with normal saline (FIGS. 2A and 2B), clarithromycin lactobinate solution (0.5% w/w) (FIGS. 2C and 2D), or a clarithromycin emulsion that comprises (0.5% w/w clarithromycin) (FIGS. 2E and 2F).

### DETAILED DESCRIPTION OF THE INVENTION

The present invention, in one aspect, provides pharmaceutical compositions for delivering macrolides. Such compositions are oil-in-water emulsions that comprise a macrolide, an oil component, an emulsifier, and water. Optionally, these compositions may further comprise a stabilizer or a tonicity modifier. The compositions of the present invention have one or more of the following properties: (1) injectable, (2) stable under appropriate storage conditions, (3) vein non-irritable, (4) containing macrolides at pharmaceutically effective concentrations, (5) sterilizable by filtration, (6) containing components acceptable by regulatory agencies (e.g., the FDA), and (7) not causing hyperlipodemia or other side effects.

An “oil-in-water emulsion” refers to a colloidal dispersion system in which liquid oil is dispersed in small droplets (the discrete phase, also referred to as “the oil phase”) in an aqueous medium (the continuous phase, also referred to as “the aqueous phase”).

In certain embodiments, greater than 60%, 65%, 70%, 75%, 80%, 85%, 90%, 92%, 94%, 95%, 96%, 97%, 98%, or 99% of a macrolide is present in the oil phase.

A “macrolide” refers to an antibiotic that contains a many-membered lactone ring to which one or more deoxy sugars are attached. Exemplary macrolides include, but are not limited to erythromycin, erythromycin estolate, erythromycin ethylsuccinate, erythromycin glucoheptonate, erythromycin lactobionate, erythromycin propionate, erythromycin stearate, clarithromycin, azithromycin, spiramycin, dirithromycine, josamycine, josamycine propionate, kitasamycine, midecamycine, miocamycine, oleandomycine phosphate, roxithromycine, spiramycine, spiramycine adipate, rovamycine, and clarithromycin.

“Clarithromycin” refers to 6-O-methyl-erythromycin (see, U.S. Pat. No. 4,331,803) with a structure shown below

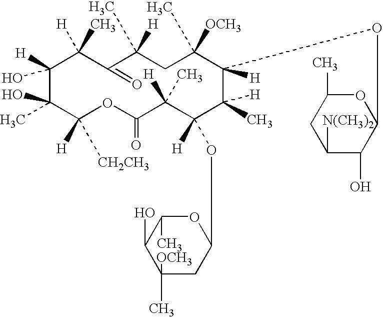

“Clarithromycin” also refers to semisynthetic derivatives of clarithromycin (e.g., pharmaceutically acceptable salts and esters of clarithromycin).

“Pharmaceutically acceptable salts and esters” refers to salts and esters which are, within the scope of sound medical judgment, suitable for use in contact with the tissues of humans and lower animals without undue toxicity, irritation, allergic response, and the like, and effective for their intended use in the chemotherapy and prophylaxis of antimicrobial infections. Among the more common pharmaceutically acceptable salts and esters of macrolide antibiotics are acetate, estolate (lauryl sulfate salt of the propionate ester), ethyl succinate, gluceptate (glucoheptonate), lactobionate, stearate, and hycrochloride forms. Other acid salts used in the pharmaceutical arts are the following: adipate, alginate, aspartate, benzoate, benzene-sulfonate, bisulfate, butyrate, citrate, camphorate, camphorsulfonate, cyclopentaneproiponate, digluconate, dodecylsulfate, ethanesulfonate, fumarate, gluconate, glycerophosphate, hemisulfate, heptaonate, hexanoate, hydrobromide, hydroiodide, 2-hydroxyethanesulfonate, lactate, maleate, methanesulfonate, 2-naphthalene-sulfonate, nicotinate, oxalate, pamoate, pantothenate, pectinate, persulfate, 3-pheylpropionate, picrate, pivalate, propionate, succinate, tartrate, thiocyanate, tosylate, and undecanoate. Basic nitrogen-containing groups can be quaternized with such agents as lower alkyl halides, such as methyl, ethyl, propyl and butyl chloride, bromides and iodides; dialkyl sulfates like dimethyl, diethyl, dibutly, and diamyl sulfates; long chain halides such as decyl, lauryl, myristyl and stearyl chlorides, bromides and iodides; aralkyl halides like benzyl and phenethyl bromides and others. Water or oil-soluble or dispersible products are thereby obtained.

“Therapeutically effective concentration” (used exchangeably with “pharmaceutically effective concentration”) refers to the concentration of a macrolide (e.g., clarithromycin) that is effective to treat or prevent susceptible bacterial or other microbial infections, at a reasonable benefit/risk ratio applicable to any medical-treatment.

Exemplary therapeutically effective concentrations of macrolides (e.g., clarithromycin) include, but are not limited to, from about 2.5 mg/mL to about 10 mg/mL. In certain embodiments, the concentration of a macrolide in an oil-in-water emulsion is at least about 1, 2, 3, 4, 5, 6, 7, 8, 9, 10, 11, 12, 13, 14, 15, 16, 17, 18, 19, 20, or 25 mg/ml. In certain embodiments, the concentration of a macrolide in an oil-in-water emulsion is at least about 0.1%, 0.2%, 0.3%, 0.4%, 0.5%, 0.6%, 0.7%, 0.8%, 0.9%,1.0%,1.2%,1.4%,1.6%,1.8%, 2.0%, 2.5%, 3%, 4%, or 5% of the total weight of the emulsion.

“Concentration by weight,” as used herein, refers to the ratio (in percentage) of the weight of a component (e.g., a macrolide) of a composition (e.g., a macrolide oil-in-water emulsion) to the total weight of the composition, if not otherwise noted.

The term “oil” is used herein in a general sense to identify hydrocarbon derivatives, carbohydrate derivatives, or similar organic compounds that are liquid at body temperatures, e.g., about 37° C., and are pharmacologically acceptable in injectable formulations. This class includes vegetable oils, animal fats, and synthetic oils, as well as various liquids that are obtained by chemical treatment of such oils and fats. In certain embodiments, oil used in the present invention does not comprise tocopherols, tocotrienols, or derivatives thereof.

The term “oil component” refers to an oil, or a combination of multiple oils in an oil-in-water emulsion.

In certain embodiments, the oil component of oil-in-water emulsions of the present invention comprises a monoglyceride, a diglyceride, a triglyceride, or a mixture thereof. In certain embodiments, the oil component comprises an ester formed between one or more fatty acids and an alcohol other than glycerol.

“Vegetable oil” refers to oil derived from plant seeds or nuts. Exemplary vegetable oils include, but are not limited to, almond oil, borage oil, black currant seed oil, corn oil, safflower oil, soybean oil, cottonseed oil, peanut oil, olive oil, rapeseed oil, coconut oil, palm oil, canola oil, etc.

Vegetable oils are typically “long-chain triglycerides,” formed when three fatty acids (usually about 14 to about 22 carbons in length, with unsaturated bonds in varying numbers and locations, depending on the source of the oil) form ester bonds with the three hydroxyl groups on glycerol. In certain embodiments, vegetable oils of highly purified grade (also called “super refined”) are generally used to ensure safety and stability of oil-in-water emulsions.

“Medium chain trglycerdes” (MCT's) is another class of triglyceride oil that can be either naturally derived or synthetic. MCT's are made from fatty acids that are usually about 6 to about 12 carbons in length. Like vegetable oils, MCT's have been used extensively in emulsions designed for injection as a source of calories, for patients requiring parenteral nutrition. Such oil is commercially available as Miglyol 812 from SASOL GmbH, Germany, CRODAMOL GTCC-PN from Croda Inc. of Parsippany, N.J., or Neobees M-5 oil from PVO International, Inc., of Boonton, N.J. Other low-melting medium chain oils may also be used in the present invention.

“Animal fat” refers to oil derived from an animal source. It also comprises triglycerides, but the lengths of, and unsaturated bonds in, the three fatty acid chains vary, compared to vegetable oils. Animal fats from sources that are solid at room temperature (such as tallow, lard, etc.) can be processed to render them liquid if desired. Other types of animal fats that are inherently liquid at room temperature include various fish oils, etc.

In certain embodiments, the combinations of vegetable oil and MCT oil are used in the present invention. Such combinations generally have long record of safe use in combination in injectable emulsions and provide the superior stability for the emulsion of this invention. The specific type of vegetable oil used (i.e., soy bean oil, corn oil, or safflower oil, etc.) is not critical, so long as it is safe, well tolerated, pharmaceutically acceptable, chemically stable and provides emulsion droplets having a desired size range.

The content of the total oil component in the macrolide emulsions of this invention may be within a range of 1% to 50%, by weight. In certain embodiments, the total concentration of the oil component is about at most about 5%, 10%, 15%, 20%, 25%, 30%, 35%, 40%, 45%, or 50% by weight. In certain embodiments, the oil-in-water emulsions comprise oil in an amount that does not result in hyperlipodemia when administered to a subject.

In certain embodiments, the vegetable oil to MCT oil ratio in an oil-in-water emulsion is within a range of about 9:1 to about 1:1, by weight. In certain embodiments, the ratio of the vegetable oil to MCT oil is abut 9:1, 8:1, 7:1, 6:1, 5:1, 4:1, 3:1, 2:1 or 1:1.

An “emulsifier” refers to a compound that prevents the separation of the injectable emulsion into individual oil and aqueous phases. Emulsifiers useful in the present invention generally are (1) compatible with the other ingredients of the oil-in-water emulsions of the present invention, (2) do not interfere with the stability or efficacy of the macrolides in the emulsions, (3) are stable and does not deteriorate in the preparation, and (4) are non-toxic.

Suitable emulsifiers include, but are not limited to, propylene glycol mono- and di-fatty acid esters, polyoxyethylene sorbitan fatty acid esters, polyoxyethylene fatty acid esters, polyoxyethylene-polyoxypropylene co-polymers and block co-polymers, salts of fatty alcohol sulphates, sorbitan fatty acid esters, esters of polyethylene-glycol glycerol ethers, oil and wax based emulsifiers, glycerol monostearate, glycerine sorbitan fatty acid esters and phospholipids.

A “phospholipid” refers to a triester of glycerol with two fatty acids and one phosphate ion. Exemplary phospholipids useful in the present invention include, but are not limited to, phosphatidyl chlorine, lecithin (a mixture of choline ester of phosphorylated diacylglyceride), phosphatidylethanolamine, phosphatidylglycerol, phosphatidic acid with about 4 to about 22 carbon atoms, and more generally from about 10 to about 18 carbon atoms and varying degrees of saturation. The phospholipid component of the drug delivery composition can be either a single phospholipid or a mixture of several phospholipids. The phospholipids should be acceptable for the chosen route of administration.

The phospholipids useful in the present invention can be of natural origin. Naturally occurring lecithin is a mixture of the diglycerides of stearic, palmitic, and oleic acids, linked to the choline ester of phosphoric acid, commonly called phosphatidylcholine, and can be obtained from a variety of sources such as eggs and soya beans. Soy lecithin and egg lecithin (including hydrogenated versions of these compounds) have a long history of safety, possess combined emulsification and solubilization properties, and tend to be broken down into innocuous substances more rapidly than most synthetic surfactants. Commercially available soya phospholipids are the Centrophase and Centrolex products marketed and sold by Central Soya, Phospholipon from Phospholipid GmbH, Germany, Lipoid by Lipoid GmbH, Germany, and EPIKURON by Degussa.

Phospholipids useful in the present invention can also be synthesized. Exemplary common synthetic phospholipids are listed below:

Diacylglycerols

1,2-Dilauroyl-sn-glycerol (DLG)
1,2-Dimyristoyl-sn-glycerol (DMG)
1,2-Dipalmitoyl-sn-glycerol (DPG)
1,2-Distearoyl-sn-glycerol (DSG)
   Phosphatidic Acids
1,2-Dimyristoyl-sn-glycero-3-phosphatidic acid, sodium salt (DMPA,Na)
1,2-Dipalmitoyl-sn-glycero-3-phosphatidic acid, sodium salt (DPPA,Na)
1,2-Distearoyl-sn-glycero-3-phosphatidic acid, sodium salt-(DSPA,Na)
   Phosphocholines
1,2-Dilauroyl-sn-glycero-3-phosphocholine (DLPC)
1,2-Dimyristoyl-sn-glycero-3-phosphocholine (DMPC)
1,2-Dipalmitoyl-sn-glycero-3-phosphocholine (DPPC)
1,2-Dipalmitoyl-sn-glycero-3-phosphocholine (DPPC)
1,2-Distearoyl-sn-glycero-3-phosphocholine (DSPC)
1,2-Distearoyl-sn-glycero-3-phosphocholine (DSPC)
   Phosphoethanolamines
1,2-Dilauroyl-sn-glycero-3-phosphoethanolamine (DLPE)
1,2-Dimyristoyl-sn-glycero-3-phosphoethanolamine (DMPE)
1,2-Dipalmitoyl-sn-glycero-3-phosphoethanolamine (DPPE)
1,2-Distearoyl-sn-glycero-3-phosphoethanolamine (DSPE)
   Phosphoglycerols
1,2-Dilauroyl-sn-glycero-3-phosphoglycerol, sodium salt (DLPG)
1,2-Dimyristoyl-sn-glycero-3-phosphoglycerol, sodium salt (DMPG)
1,2-Dimyristoyl-sn-glycero-3-phospho-sn-1-glycerol, ammonium salt (DMP-sn-1-G,NH4)
1,2-Dipalmitoyl-sn-glycero-3-phosphoglycerol, sodium salt (DPPG,Na)
1,2-Distearoyl-sn-glycero-3-phosphoglycerol, sodium salt (DSPG,Na)
1,2-Distearoyl-sn-glycero-3-phospho-sn-1-glycerol, sodium salt (DSP-sn-1G,Na)
   Phosphoserines
1,2-Dipalmitoyl-sn-glycero-3-phospho-L-serine, sodium salt (DPPS,Na)
   Mixed Chain Phospholipids
1-Palmitoyl-2-oleoyl-sn-glycero-3-phosphocholine (POPC)
1-Palmitoyl-2-oleoyl-sn-glycero-3-phosphoglycerol, sodium salt (POPG,Na)
1-Palmitoyl-2-oleoyl-sn-glycero-3-phosphoglycerol, ammonium salt (POPG,NH4)
   Lysophospholipids
1-Palmitoyl-2-lyso-sn-glycero-3-phosphocholine (P-lyso-PC)
1-Stearoyl-2-lyso-sn-glycero-3-phosphocholine (S-lyso-PC)
   Pegylated Phospholipids
N-(Carbonyl-methoxypolyethyleneglycol 2000)-MPEG-2000-DPPE
1,2-dipalmitoyl-sn-glycero-3-phosphoethanolamine, sodium salt
N-(Carbonyl-methoxypolyethyleneglycol 5000)-MPEG-5000-DSPE
1,2-distearoyl-sn-glycero-3-phosphoethanolamine, sodium salt
N-(Carbonyl-methoxypolyethyleneglycol 5000)-MPEG-5000-DPPE
1,2-dipalmitoyl-sn-glycero-3-phosphoethanolamine, sodium salt
N-(Carbonyl-methoxypolyethyleneglycol 750)-MPEG-750-DSPE
1,2-distearoyl-sn-glycero-3-phosphoethanolamine, sodium salt
N-(Carbonyl-methoxypolyethyleneglycol 2000)-MPEG-2000-DSPE
1,2-distearoyl-sn-glycero-3-phosphoethanolamine, sodium salt

The amount of phospholipids, by weight, in the emulsions of the present invention may be within a range of about 1.2% to about 5%. In certain embodiments, the phospholipids in the emulsions are at a concentration, by weight, about 1.2%, 1.5%, 2%, 2.5%, 3%, 3.5%, 4%, 4.5%, or 5%.

The compositions of the present invention may optionally contain additives (referred to as “tonicity modifiers”) to adjust tonicity of the emulsion. Such compounds may be glycerol (1-5% by weight) and amino acids (1-5% by weight). In certain embodiments, the concentration of a tonicity modifier is about 1%, 1.5%, 2%, 2.5%, 3%, 3.5%, 4%, 4.5%, or 5%.

The compositions of the present invention may optionally contain stabilizing agents (referred to as “stabilizers”) to prevent or reduce the deterioration of the other components in oil-in-water emulsions, including antioxidants (e.g., glycine, α-tocopherol or ascorbate), or to prevent or inhibit microbial growth in the emulsions (e.g., EDTA). In certain embodiments, the concentration of glycine is about 0.1% to about 5% (e.g., about 1%) by weight. In certain embodiments, the concentration of EDTA is about 0.001% to about 0.01% (e.g., about 0.005%) by weight.

In certain embodiments, the oil-in-water emulsions of the present invention may comprise a compound (e.g., a fatty acid or N-methyl pyrrolidone) to increase the solubility of a macrolide in the oil phase of the emulsions, and to prevent the precipitation of the macrolide out of the emulsion. In other embodiments, although the oil-in-water emulsions may contain the compound as described above, the stability and/or the ability of the emulsion of this invention to deliver a therapeutically effective concentration of a macrolide does not require the presence of such a compound.

The aqueous phase of an oil-in-water emulsion of the present invention is usually at a concentration of at least about 70% by weight of the emulsion composition. In certain embodiments, the aqueous phase is at a concentration of at least about 75%, 80% or 85%, by weight of the emulsion composition.

In certain embodiments, some or all of the components other than the macrolide in the oil-in-water emulsion (e.g., an oil component, an emulsifier, a stabilizer, and a tonicity modifier) is safe, well tolerated, and acceptable by the FDA for intravenous injection.

A component of oil-in-water emulsions is. regarded as “safe” if it does not cause undesired systemic reactions such as anaphylactic shock in patients.

A component of oil-in-water emulsions is regarded as “well tolerated” if it does not result in substantially adverse effects at the injection site, such as phlebitis, vein inflammation or vein irritation.

A component of oil-in-water emulsions is regarded as “acceptable by the FDA” if it has been used in intravenous injection products approved by the FDA as of the filing date of the present application, and is being used at a concentration comparable to those used in FDA approved products.

In certain embodiments, some or all of the components other than the macrolide in the oil-in-water emulsion (e.g., an oil component, an emulsifier, a stabilizer, and a tonicity modifier) is generally regarded as safe for use in intravenous injections by a drug regulatory authority.

A component of oil-in-water emulsion is “generally regarded as safe for use in intravenous injections by a drug regulatory authority” if it has been used in intravenous injection products approved by the FDA or a drug regulatory authority in Europe as of the filing date of the present application, and is being used at a concentration comparable to those used in the products approved by the FDA in the United States or by a drug regulatory authority in Europe.

In certain embodiments, the oil-in-water emulsions of the present invention are vein non-irritable. “Vein non-irritable” refers to the property of a compound or composition, when administered intravenously, does not cause substantial irritation at the injection site, as evident by, for example, thickened skin, necrotic skin, local redness, local swelling, venous dilation with blood clog formation, or venous embolism with subcutaneous inflammation.

In certain embodiments, the oil-in-water emulsions of the present invention are stable both- chemically and physically. An oil-in-water emulsion is “physically stable” if it may be stored under appropriate conditions for at least 1 month without increase in average droplet size by more than 100%, or evidence of phase separation or oil droplet aggregation (coalescence). In certain embodiments, the average size of oil droplets of an emulsion of the present invention does not increase by more than about 10%, 20%, 25%, 30%, 40%, 50%, 75%, 100%, 125%, 150%, 175%, or 200% under appropriate storage conditions for at least 1, 2, 3, 4, 5, 6, 9, 12, 15, 18, or 24 months.

An oil-in-water emulsion is “chemically stable” if the macrolide concentration in the emulsion does not change by about 20% under appropriate storage conditions for at least 1 month. In certain embodiments, the macrolide concentration in an emulsion of the present invention does not change by about 5%, 10%, 15% or 20% under appropriate storage conditions for at least 1, 2, 3, 4, 5, 6, 9, 12, 15, 18, or 24 months.

In certain embodiments, the oil droplets of the oil-in-water emulsions are of sub-micron size. A “sub-micron size droplet” refers to an oil droplet in an oil-in-water emulsion having an average diameter of less than 1 micron as measured by conventional sizing techniques such as laser light scattering spectrometry. In certain embodiments, the oil droplets of the compositions of the present invention have an average diameter of less than 500, 450, 400, 350, 300, or 250 nm. Oil droplets of sub-micron size are desired for the safe passage of these droplets in, the capillary blood vessel in the circulation. Droplets of greater than 5 micron in diameter are believed to be unsafe for intravenous injection since they may block the capillary blood vessel resulting in pulmonary embolism. In certain embodiments, the oil droplets of the compositions of the present invention have an average diameter of less than 0.2-micron (200 nm) so that the emulsion may be sterilized by filtering through a 0.2 micron sized filter membrane. In certain embodiments, the oil droplets of the compositions of the present invention have an average diameter of less than about 150,100, 75, 50, 25, 20,15, or 10 nm.

In certain embodiments, the oil-in-water emulsions of the present invention have a wide range of temperature stability (e.g., −20° C. to 40° C.). In certain embodiments, the oil-in-water emulsions are stored at about 5° C. to about 25° C., or about 2° C. to about 8° C.

In certain embodiments, the oil-in-water emulsions are vein non-irritable, stable and capable of delivering pharmaceutically effective amount of macrolides. Such emulsions may comprise (a) a macrolide at a concentration of at least 0.5% by weight, (b) an oil component at a concentration of at most 10% by weight, (c) one or more phospholipids at a total concentration between about 1.2% to about 5% by weight, and (d) water. These emulsions may further comprise one or more stabilizers and/or tonicity modifiers.

Exemplary oil-in-water emulsions that are both vein non-irritable and stable comprise: (a) clarithromycin at a concentration of about 0.5% or higher by weight, (b) Miglyol 812 (or another medium chain triglyceride) at a concentration of about 1% to about 5% by weight, (c) soybean oil (or another vegetable oil) at a concentration of about 5% to about 9% by weight, (d) phospholipon 90G (or another phospholipid or phospholipids) at a concentration of about 3% by weight, and (e) water, and may optionally comprise one or more of the following components: (i) glycine at a concentration of about 1%, (ii) glycerol at a concentration of about 1.5%, and (iii) EDTA at a concentration of about 0.005%.

Other exemplary oil-in-water emulsions that are both vein non-irritable and stable may comprise: (a) clarithromycin at a concentration of about 0.5% or higher by weight, (b) Miglyol 812 (or another medium chain triglyceride) at a concentration of about 1% to about 5% by weight, (c) soybean oil (or another vegetable oil) at a concentration of about 5% to about 9% by weight, (d) egg lecithin (e.g., Lipoid E-80) at a concentration of about 3% by weight, and (e) water, and may optionally comprise one or more of the following components: (i) glycine at a concentration of about 1%, and (ii) glycerol at a concentration of about 1.5%.

Other exemplary oil-in-water emulsions that are both vein non-irritable and stable may comprise: (a) clarithromycin at a concentration of about 0.5% or higher by weight, (b) soybean oil (or another vegetable oil) at a concentration of about 5% to about 10% by weight, (c) egg lecithin (e.g., Lipoid E-80) or soy lecithin at a concentration of about 3% by weight, and (d) water, and may optionally comprise one or more of the following components: (i) glycine at a concentration of about 1%, and (ii) glycerol at a concentration of about 1.5%.

Other exemplary oil-in-water emulsions that are both vein non-irritable and stable may comprise: (a) clarithromycin at a concentration of about 0.5% or higher by weight, (b) soybean oil (or another vegetable oil) at a concentration of about 5% to about 10% by weight, (d) egg lecithin (e.g., Lipoid E-80) or a soy lecithin at a concentration of about 1.2% by weight, and (e) water, and may optionally comprise one or more of the following components: (i) glycine at a concentration of about 1%, and (ii) glycerol at a concentration of about 1.5%.

Further exemplary oil-in-water emulsions that are both vein non-irritable and stable may comprise: (a) clarithromycin at a concentration of about 0.5% or higher by weight, (b) soybean oil (or another vegetable oil) at a concentration of about 2.5% to about 5% by weight, (d) egg lecithin (e.g., Lipoid E-80) or a soy lecithin at a concentration of about 1.2% by weight, and (e) water, and may optionally comprise one or more of the following components: (i) glycine at a concentration of about 1%, and (ii) glycerol at a concentration of about 1.5%.

Additional exemplary oil-in-water emulsions that are both vein non-irritable and stable may comprise: (a) erythromycin at a concentration of about 0.5% or higher by weight, (b) Miglyol 812 (or another medium chain triglyceride) at a concentration of about 1% to about 5% by weight, (c) soybean oil (or another vegetable oil) at a concentration of about 5% to about 9% by weight, (d) egg lecithin (e.g., Lipoid E-80) at a concentration of about 3% by weight, and (e) water, and may optionally comprise one or more of the following components: (i) glycine at a concentration of about 1%, and (ii) glycerol at a concentration of about 1.5%.

The present invention also provides methods for preparing macrolide (e.g., clarithromycin) emulsion compositions described herein. Such emulsion compositions may be prepared by (a) forming a mixture that comprises (i) a pharmaceutically effective amount of a macrolide free base, (ii) an oil component (e.g., a vegetable oil, or a combination of a vegetable oil and a medium chain triglyceride), and (iii) a phospholipid, (b) forming an oil-in-water emulsion with the mixture of step (a) and an aqueous solution, (c) adjusting the pH of the emulsion of step (b) to about 2-5, and (d) re-adjusting the pH of the emulsion resulting from step (c) to about 6-8 to provide an injectable oil-in-water emulsion that contains a pharmaceutically effective amount of the macrolide.

In certain embodiments, step (a) may be performed by dissolving the macrolide in a solution (e.g., alcohol) and mixing the dissolved macrolide with a composition that comprises the oil component (e.g., a vegetable oil, or a combination of a vegetable oil and a medium chain triglyceride) and the phospholipid. The alcohol component (e.g., ethanol) used in solubilizing the macrolide is an intermediate, and is usually removed to a residual amount of less than 5% (w/w) after step (a), such as by using a rotary evaporator. The amount of alcohol required depends on the need to completely solubilize the macrolide.

In certain embodiments, step (b) may be performed by adding the aqueous solution to the mixture of step (a) to form a primary emulsion. The aqueous solution may be water or a buffer solution, and may contain stabilizer(s) and/or tonicity modifier(s). The formation of the primary emulsion may be performed or facilitated by the use of mechanical homogenization (e.g., high shear mixing, high pressure extrusion, and microfluidization) or other suitable techniques.

In certain embodiments where the pH of the primary emulsion is neutral (e.g., pH 6-8), some macrolides (e.g., clarithromycin) may partially become crystallized and precipitate out of the emulsion. The crystallized macrolide may be re-dissolved into the emulsion if the pH of the emulsion is adjusted to be acidic (e.g., about 2-4, about 3-4, about 3-5, or about 2-5) by, for example, HCl. After the re-dissolution of the crystallized macrolide, the pH of the emulsion may be re-adjusted to be neutral (e.g., about 6-7 or about 6-8) by, for example, NaOH. The neutralization of the emulsion usually does not cause the macrolide to re-precipitate out of the emulsion. Accordingly, the above steps of first adjusting pH of the emulsion to become acidic and then readjusting pH of the emulsion to be neutral allow for a higher concentration of the macrolide in the oil-in-water emulsion.

The above-described emulsion may be further refined by cycling through a microfluidizer homogenizer or a similar apparatus to obtain a stable emulsion having fairly uniform oil droplet sizes. The resulting refined emulsion may be filter sterilization, for example, through a 0.22-micron sterile filter.

Besides being ready-to-use oil-in-water emulsions, the macrolide compositions of the present invention can also be prepared with a cryoprotectant(s) as-a lyophilized solid, i.e., “an oil-in-solid dispersion system” that can be reconstituted at a later date and diluted with water to reform the oil-in-water emulsion before injection.

As used herein, the term “an oil-in-solid dispersion system” refers to a solid matrix prepared by freeze-drying (lyophilizing) an oil-in-water emulsion of the present invention, which can reform an oil-in-water emulsion of similar droplet size upon mixing with water (reconstitution). In certain embodiments, the average droplet size of the reformed emulsion is no more than about 500%, 400%, 300%, 200%, or 150% of the average droplet size of the emulsion before the freeze-drying. An oil-in-solid dispersion system of this invention may be optionally prepared by spray drying.

“Cryoprotectants” used in the emulsion compositions of the present invention refers to those ingredients which are added to maintain the discrete and submicron droplets of the emulsion during the freeze-drying process and, upon the removal of water of the emulsion, to provide a solid matrix for the droplets to form the an oil-in-solid dispersion system.

Cryoprotectants that may be used in the emulsion compositions of this invention include, but are not limited to, polyols, monosaccharides, disaccharides, polysaccharides, amino acids, peptides, proteins, and hydrophilic polymers, or mixtures thereof.

Polyols that may be used in the present invention include, but are not limited to, glycerin, mannitol, erythritol, maltitol, xylitol, sorbitol, polyglycitol or mixtures thereof.

Monosaccharides that may be used in this invention include, but are not limited to, glucose, mannose, fructose, lactulose, allose, altrose, gulose, idose, galactose, talose, ribose, arabinose, xylose, lyxose or mixtures thereof.

Disaccharides that may be used in this invention include, but are not limited to, sucrose, lactose, maltose, isomaltose, trehalose, cellubiose or mixtures thereof.

Polysaccharides that may be used in this invention include, but are not limited to, cellulose, amylose, inulin, chitin, chitosan, amylopectin, glycogen, pectin, hyaruronic acid or mixtures thereof.

Amino acids that may be used in this invention include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine or mixtures thereof.

Peptides that may be used in this invention include, but are not limited to, diglycine and triglycine.

Proteins that may be used in this invention include, but are not limited to, albumin, collagen, casein, and gelatin.

Hydrophilic polymers that may be used in this invention include, but are not limited to, polyethylene glycols povidones, poloxamers, polyvinyl alcohols or mixtures thereof. The most preferred hydrophilic polymers are polyethylene glycols and povidones.

The concentration of the cryoprotectants used in the liquid emulsion compositions may be in the range of about 2% to about 40% w/w, such as about 5% to about 20% w/w and about 10% to about 15% w/w.

The macrolide formulations of the present invention may be used to treat bacterial and/or other microbial infections for which microlides are effective, including upper and lower respiratory tract infections, skin infections, atypical mycobacterial infections and Helicobacter pylori infection. The macrolide formulations of the present invention may be administered to a subject (e.g., human or other mammals) in need thereof at a pharmaceutically effective amount by various routes, including but not limited to, intravenous, intramuscular, intra-arterial, intrathecal, intraocular, subcutaneous, intraarticular and intra-peritoneal administration.

“Pharmaceutically effective amount” refers to an amount of a macrolide oil-in-water emulsion that is sufficient in treating bacterial and/or other microbial infections.

The following examples are intended to illustrate the invention without limiting the practice thereof.

### EXAMPLES

### Example 1

This example provide a method for preparing injectable clarithromycin emulsion compositions that comprise an oil component (e.g., a mixture of MCT (Miglyol 812, EP by SASOL) and soybean oil of high purity (USP and Super-refined by Croda)), a soy lecithin phospholipid (e.g., phospholipon 90G, a soy lecithin containing about 90% wt. phosphotidylcholine by Phospholipid GmbH) as emulsifier, glycine and glycerol as stabilizer/tonicity agents, and water.

Clarithromycin was first dissolved in a combination of Miglyol 812 and soybean oil, phospholipon 90G, and ethanol to form a clarithromycin solution at 25° C., using conventional equipment such as a sonicator. The solution was then subject to rotary evaporation to reduce ethanol to a residual amount of less than 5% w/w to form an oil phase. Appropriate amount of an aqueous phase containing glycine and glycerol was added to the oil phase to produce a primary o/w emulsion by high shear mixing (Ultra-Turrax, Model SDT1810, by Tekmar Company). The pH of the primary emulsion was adjusted to pH 2-5 with HCl and then readjusted to neutral (pH 6-8) with a NaOH solution. The clarithromycin primary emulsion was then cycled through a high-pressure homogenizer (Microfluidizer Model M110F by Microfluidics, MA) to produce a fine emulsion with desired oily droplet size that was filter sterilized through a 0.22-micron filter.

Table 1.1 describes a fine clarithromycin emulsion composition of the 5 mg/g clarithromycin concentration using methods disclosed in this invention.

TABLE 1.1

| Component | % by weight |
|---|---|
| Clarithromycin | 0.5 |
| Miglyol 812 | 5.0 |
| Soybean Oil, high purity | 5.0 |
| Phospholipon 90G | 3.0 |
| Glycine | 1.0 |
| Glycerol | 2.5 |

HCl/NaOH, to adjust pH
Water, to add to the final wt

### Example 2

The stability results of the emulsion described in Example 1 are shown in Table 2.1. The average droplet diameters were determined using a dynamic light scattering particle sizer (Model 370 Submicron Particle Sizer by Particle Sizing System, Santa Barbara, Calif.). Counts of particulates or droplets of greater than 5 microns were obtained using an optical microscope and hemacytometer (Bright-Line by Hausser Scientific, PA).

TABLE 2.1

| | | Average droplet diameter (nm) | | |
|---|---|---|---|---|
| | | Large droplets or particulates (>5 micron) | | |
| | | Average | | |
| | | diameter | Particulates | |
| Time | Temp (° C.) | (nm) | (>5 micron) | Appearance |
| 0 | 25 | 166.7 | No | White and uniform |
| | | 174.3 | No | White and uniform |
| | | 168.7 | No | White and uniform |
| 1 Month | −20 | 285.9 | No | White and uniform |
| | 2-8 | 168.4 | No | White and uniform |
| | 25 | 118.9 | No | White and uniform |
| | 40 | 183.8 | Yes | Large droplets |
| 2 Month | −20 | 214.7 | No | White and uniform |
| | 2-8 | 170.6 | No | White and uniform |
| | 25 | 168.0 | No | White and uniform |
| | 40 | 164.2 | Yes | Large droplets |

Stability results of clarithromycin in the emulsion are shown in Table 2.2. The Clarithromycin concentrations in the emulsion were determined by a reversed phase high-pressure liquid chromatography (Hewlett Parkard Model 1050 HPLC).

TABLE 2.2

| | | Clarithromycin |
|---|---|---|
| Time | Temp (° C.) | Concentration (mg/mL) |
| 0 | 25 | 5.00 |
| 1 Month | −20 | 5.82 |
| | 2-8 | 5.28 |
| | 25 | 4.88 |
| | 40 | 5.27 |
| 2 Month | −20 | 5.00 |
| | 2-8 | 5.08 |
| | 25 | 5.35 |
| | 40 | 4.05 |

### Example 3

The emulsion prepared according to Example 1 did not show any sign of vein irritation or inflammation following 3 consecutive days of fast infusion at 3, 4 and 5 mg/mL clarithromycin concentration at 3 times of an adult human dose (adjusted based on body weight) into rabbit marginal ear veins using the rabbit ear test method.

### Example 4

The objective of this study was to evaluate the long-term stability of an injectable clarithromycin emulsion.

A batch (400 mL) of clarithromycin emulsion was prepared to contain 5 mg/mL clarithromycin free base and other injectable ingredients as described in Example 1. The emulsion was sterilized by filtration through a 0.2-micron membrane filter. The final product was stored in type-1 glass bottles sealed with rubber closures and the bottles were placed in 5° C., 25° C. and 40° C. stability chambers. At each sampling time point, emulsion samples were removed and tested for clarithromycin concentration by HPLC, average droplet size by a laser light scattering particle sizer, and large-sized droplets by optical microscope.

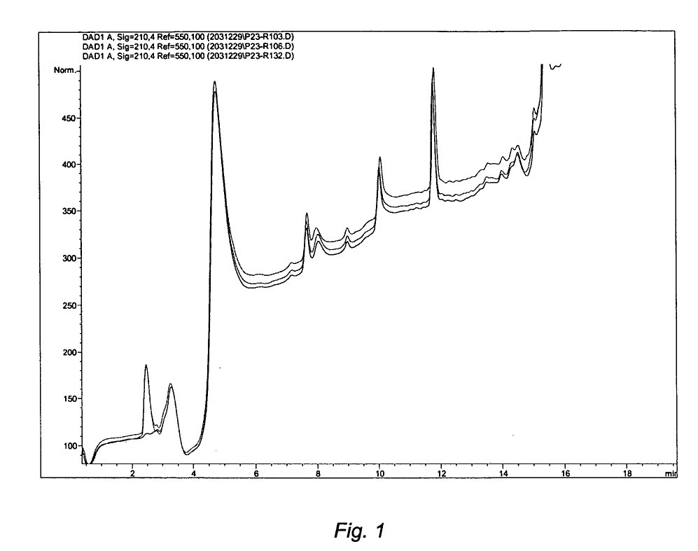

After 14-month storage at 5° C., the clarithromycin concentration in the emulsion remained unchanged; the average droplet size was maintained at about 140-170 nm in diameter and no large-sized droplets (>5 microns in diameter) were observed. Stability data are provided in the following tables and FIG. 1.

The stability prognosis of the emulsion was shown to be acceptable. It can be predicted that the clarithromycin emulsion for injection could provide a shelf life of at least 1-1.5 years at 5° C.

TABLE 4.1
Clarithromycin Concentration in Emulsion (mg/mL) by HPLC

Temperature

| Elapsed Time | −20° C. | 5° C. | 25° C. | 40° C. |
|---|---|---|---|---|
| 2.5 M | 4.6 | 4.6 | 4.5 | 4.2 |
| 3.5 M | 4.6 | 4.6 | 4.4 | 3.9 |
| 4.5 M | 4.7 | 4.6 | 4.4 | 3.5 |
| 7.5 M | 4.7 | 4.7 | 4.3 | 1.1 |
| 14 M | 4.9 | 4.7 | NA | NA |

TABLE 4.2
Clarithromycin Concentration Recovery Expressed as Percent (%)

Temperature

| Elapsed Time | −20° C. | 5° C. | 25° C. | 40° C. |
|---|---|---|---|---|
| 2.5 M | 100.0 | 100.2 | 97.0 | 91.4 |
| 3.5 M | 99.9 | 99.4 | 94.4 | 84.5 |
| 4.5 M | 101.1 | 99.9 | 94.3 | 75.0 |
| 7.5 M | 102.9 | 101.6 | 92.8 | 23.7 |
| 14 M | 106.5 | 101.6 | NA | NA |

TABLE 4.3
Average Emulsion Droplet Diameter (nm)
by Laser Light Scattering (LLS)

Temperature

| Elapsed Time | −20° C. | 5° C. | 25° C. | 40° C. |
|---|---|---|---|---|
| 2.5 M | 286 | 168 | 169 | 184 |
| 3.5 M | 215 | 171 | 168 | 164 |
| 4.5 M | 206 | 240* | 212* | 4210* |
| 7.5 M | 206 | 165 | 167 | 174 |
| 14 M | NA | 145 | NA | NA |

*Increased droplet size was due to measurements made following sample freezing at −20° C.

TABLE 4.4
Large-sized Droplet Observation (>5.0 μm in diameter)
by Optical Microscope

| | |
|---|---|
| Elapsed | Temperature |

| Time | −20° C. | 5° C. | 25° C. | 40° C. |
|---|---|---|---|---|
| 2.5 M | None | None | None | None |
| 3.5 M | None | None | None | None |
| 4.5 M | None | None | None | Populated |
| 7.5 M | None | None | None | None |
| 14 M | NA | None | NA | NA |

FIG. 1 shows representative chromatograms of clarithromycin. The peak eluted at about 12 minutes is from clarithromycin. From bottom up: clarithromycin standard solution at 0.103 mg/mL; clarithromycin emulsion sample at Time 0; and clarithromycin emulsion sample after being stored at 5° C. for 7.5 months.

### Example 5

This study was to evaluate local irritation by intravenous injection of two clarithromycin (CLM) formulations; namely, CLM emulsion for intravenous injection (CEII) and a CLM lactobionate solution for injection (CSI) using rabbit marginal vein model. CEII is identical to the clarithromycin emulsion described in Example 1 except that CEII contains 1.5% glycerol (not 2.5% as in Example 1) and additionally 0.005% edetate disodium dehydrate (U.S.P.). CSI simulates the Klaricid® solution for injection, which is an IV product marketed by Abbott Labs in UK, and contains 0.5% (w/w) CLM lactobionate.

Twelve (6 males and 6 females) New Zealand white rabbits (Oryctolagus) were randomly divided into 3 groups of 2 male and 2 female rabbits. Each rabbit was infused at a constant rate (1.0 mL/min) through marginal ear vein with CEII, CSI or normal saline followed by appearance observations daily for venous irritation reactions near the injection site and pathology examination after 3 days. CEII group (n=4): CEII at 4.39 mg/mL was infused at a dose of 30 mL/animal/day for 3 days; CSI group (n=4): CSI at 4.74 mg/mL was infused at a dose of 30 mL/animal/day for 3 days; Control group (n=4): 0.9% sodium chloride for injection was infused at a dose of 30 mL/animal/day for 3 days. Pathology examination was conducted at 48 h after the last injection. Histology specimens of the marginal ear vein were taken 2 cm downstream from the injection site and were stained with HE stain.

In the CSI group, 24 hours after the first injection, severe ear vein irritations were observed with thickened and necrotic skin accompanied by local redness and swelling in 3 of 4 rabbits, and no evidence of vein irritation seen in the 4th rabbit. The CEII group did not exhibit any signs of vein irritation along the marginal ear veins, and no difference in appearance was observed between the CEII group and the control group.

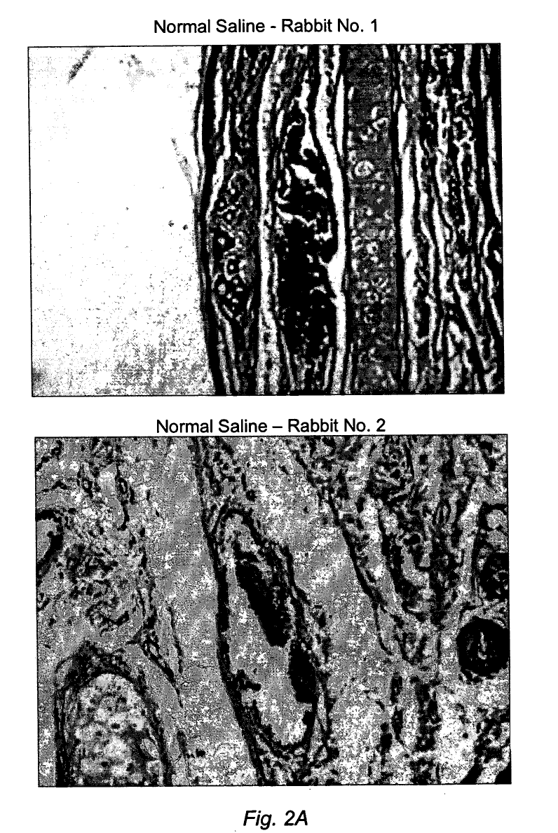

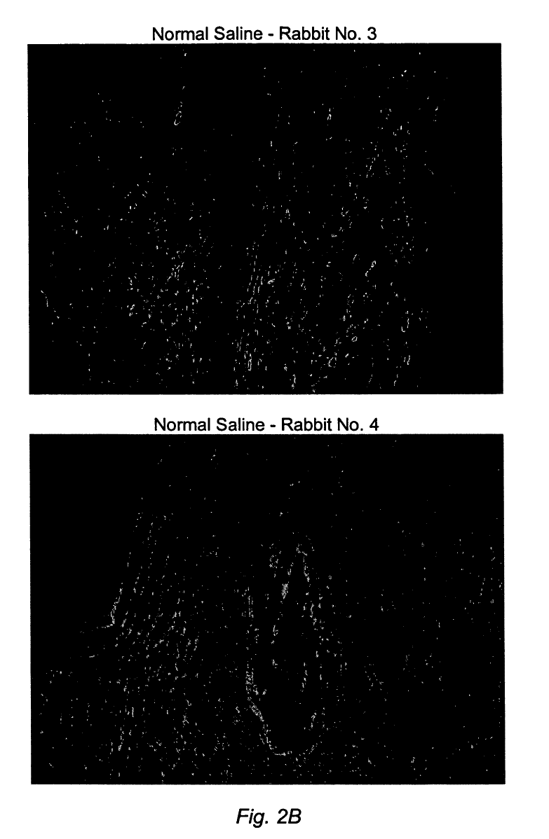

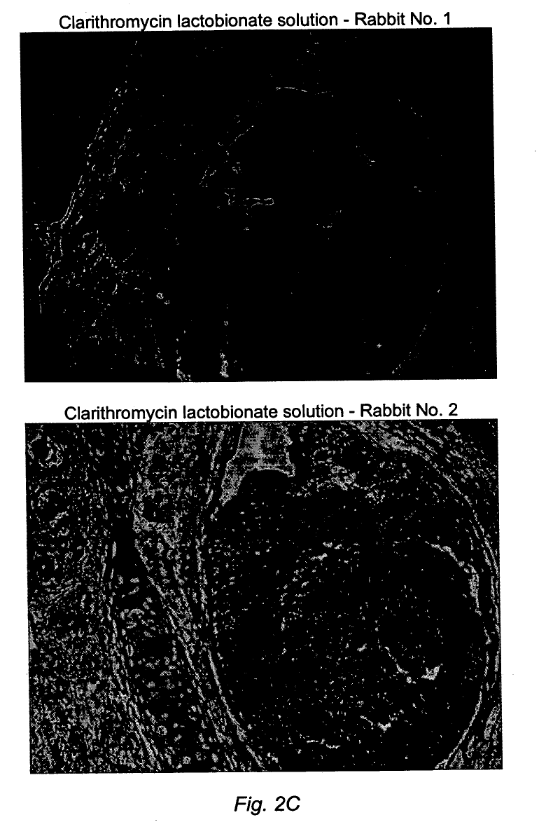

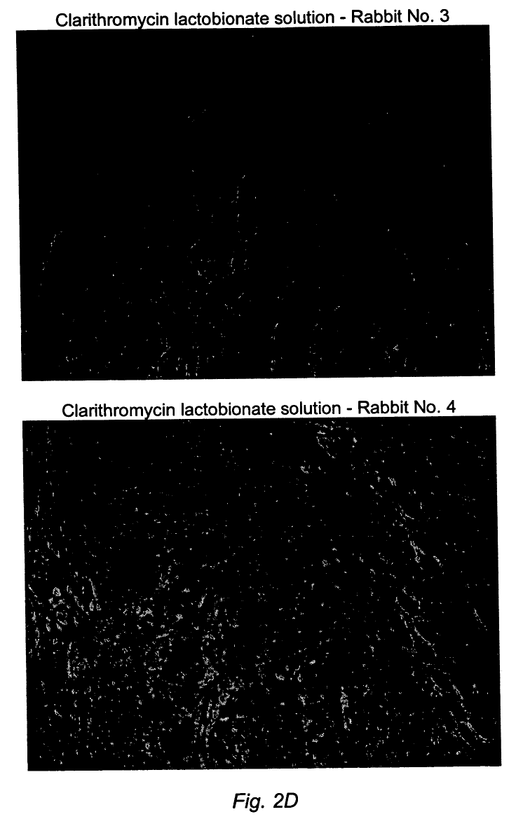

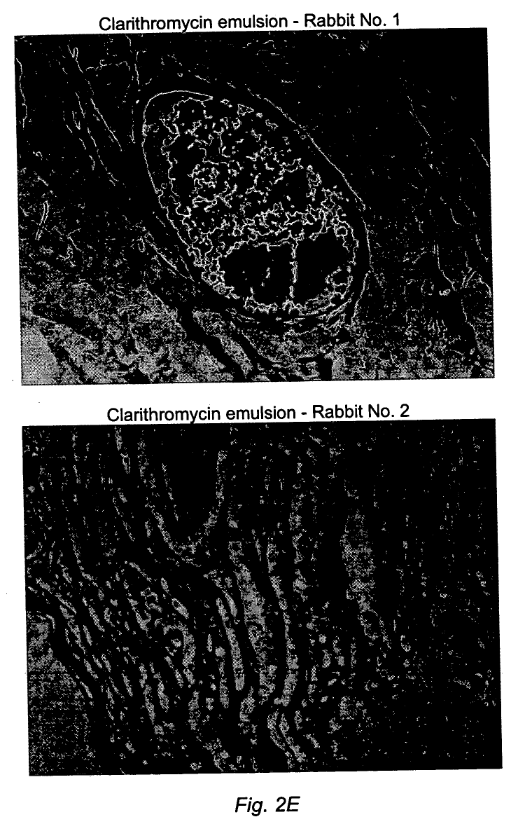

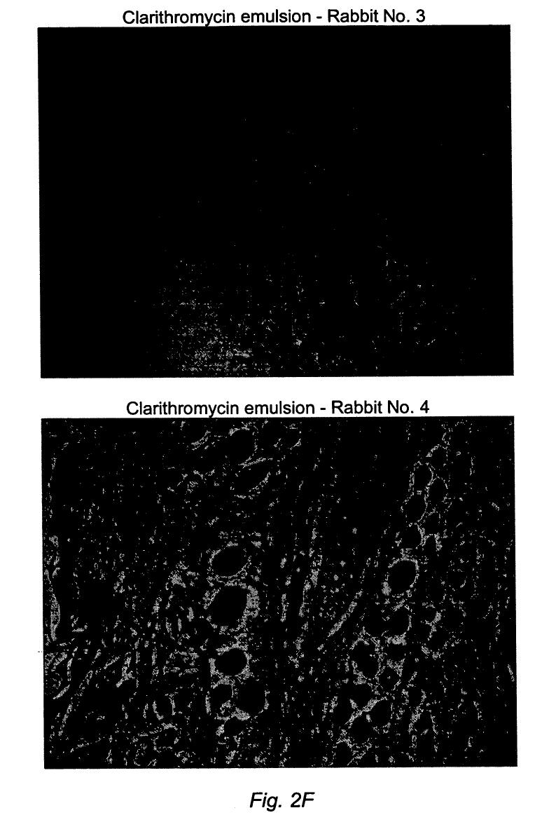

Venous dilation with blood clog formation was observed in all 4 CSI rabbits. Venous embolism with partial subcutaneous inflammation was seen in 2 of 4 CSI rabbits. No evidence of irritation to the venous endothelium was seen in the CEII and the control groups. Histology slices are shown in FIGS. 2A-2F.

This study shows that CLM lactobionate solution for injection produced severe vein irritation, while CLM emulsion for injection exhibited the same venous compatibility as the normal saline without vein irritation.

### Example 6

This example provides a method for preparing injectable erythromycin emulsion compositions that comprise an oil component (e.g., a mixture of MCT (Miglyol 812, EP by SASOL) and soybean oil of high purity (USP and Super-refined by Croda)), a soy lecithin phospholipid (e.g., phospholipon 90G, a soy lecithin containing about 90% wt. phosphotidylcholine by Phospholipid GmbH) as emulsifier, glycine and glycerol as stabilizer/tonicity agents, and water.

Erythromycin (freebase) is first dissolved in a combination of Miglyol 812 and soybean oil, phospholipon 90G, and ethanol to form an erythromycin solution at 25° C., using conventional equipment such as a sonicator. The solution is then subject to rotary evaporation to reduce ethanol to a residual amount of less than 5% w/w to form an oil phase. Appropriate amount of an aqueous phase containing glycine and glycerol is added to the oil phase to produce a primary o/w emulsion by high shear mixing. The pH of the primary emulsion is adjusted to pH 2-5 with HCl and then readjusted to neutral (pH 6-8) with a NaOH solution. The erythromycin primary emulsion is then cycled through a high-pressure homogenizer (Microfluidizer Model M110F by Microfluidics, MA) to produce a fine emulsion with desired oily droplet size that is filter sterilized through a 0.22-micron filter.

Table 6.1 describes an emulsion composition of the 5 mg/g erythromycin concentration using methods disclosed in this invention.

TABLE 6.1

| Component | % by weight |
|---|---|
| Erythromycin | 0.5 |
| Miglyol 812 | 5.0 |
| Soybean Oil, high purity | 5.0 |
| Phospholipon 90G | 3.0 |
| Glycine | 1.0 |
| Glycerol | 1.5 |

HCl/NaOH, to adjust pH
Water, to add to the final weight

### Example 7

This example provides a method for preparing injectable clarithromycin emulsion compositions that comprise soybean oil of high purity (USP and Super-refined by Croda), an egg lecithin phospholipid (e.g., Lipoid E-80 by Lipoid GmbH) as emulsifier, glycine and glycerol as stabilizer/tonicity agents, and water.

Clarithromycin (freebase) is first dissolved in a combination of soybean oil, egg lecithin, and ethanol to form a solution at 25° C., using conventional equipment such as a sonicator. The ethanolic solution is then subject to rotary evaporation to reduce ethanol to a residual amount of less than 5% w/w to form an oil phase. Appropriate amount of an aqueous phase containing glycine and glycerol is added to the oil phase to produce a primary o/w emulsion by high shear mixing. The pH of the primary emulsion is adjusted to pH 2-5 with HCl and then readjusted to neutral (pH 6-8) with a NaOH solution. The clarithromycin primary emulsion is then cycled through a high-pressure homogenizer (Microfluidizer Model M110F by Microfluidics, MA) to produce a fine emulsion with desired oily droplet size that is filter sterilized through a 0.22-micron filter.

Table 7.1 describes a clarithromycin emulsion composition of the 5 mg/g clarithromycin concentration using methods disclosed in this invention.

TABLE 7.1

| Component | % by weight |
|---|---|
| Clarithromycin | 0.5 |
| Soybean Oil, high purity | 10.0 |
| Egg lecithin (Lipoid E-80) | 3.0 |
| Glycine | 1.0 |
| Glycerol | 1.5 |

HCl/NaOH, to adjust pH
Water, to add to the final weight

### Example 8

This example provides a method to lyophilize an injectable clarithromycin emulsion composition that comprise soybean oil, medium chain triglyceride, soy lecithin phospholipid emulsifier, sucrose as a cryoprotectant and water (Table 8.1)

TABLE 8.1

| Component | % by weight |
|---|---|
| Clarithromycin | 0.5 |
| Soybean Oil, high purity | 5.0 |
| Medium chain triglyceride | 5.0 |
| Soy lecithin | 3.0 |
| Sucrose | 15.0 |

HCl/NaOH, to adjust pH
Water, to add to the final weight

Clarithromycin (freebase) is first dissolved in a combination of soybean oil, medium chain triglyceride, soy lecithin, and ethanol to form a clarithromycin solution using conventional equipment such as a sonicator. The ethanol solution is then subject to rotary evaporation to reduce ethanol to a residual amount of less than 5% w/w to form an oil phase. Appropriate amount of an aqueous phase containing sucrose is added to the oil phase to produce a primary o/w emulsion by high shear mixing. The pH of the primary emulsion is adjusted to pH 2-5 with HCl and then readjusted to neutral (pH 6-8) with a NaOH solution. The clarithromycin primary emulsion is then cycled through a high-pressure homogenizer (Microfluidizer Model M110F by Microfluidics, MA) to produce a fine emulsion with desired oily droplet size that is filter sterilized through a 0.22-micron filter. The filtered emulsion is filled into glass vials and lyophilized using a programmed lyophilization cycle, which directs the lyophilizer to reach a condenser temperature of about −80° C., a shelf temperature of about −40° C., chamber vacuum of about 50 milliTorr. The dried emulsion is then sealed in the glass vial with a rubber stopper with nitrogen gas filled in the head space. Such dried emulsion can be re-hydrated to form an oil-in-water emulsion described herein.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An injectable oil-in-water emulsion comprising:
(a) a macrolide at a concentration of at least about 0.5% by weight,
(b) a vegetable oil at a concentration of at most 10% by weight,
(c) one or more phospholipids at a total concentration between about 1.2% to about 5% by weight, and
(d) water.

2. The injectable oil-in-water emulsion of claim 1 further comprising a medium chain triglyceride, wherein
(i) the total concentration of the vegetable oil and the medium chain triglyceride is at most 10% by weight, and
(ii) the weight ratio of the vegetable oil to the medium chain triglyceride is between about 9:1 to about 1:1.

3. The injectable oil-in-water emulsion of claim 1 wherein the macrolide is clarithromycin having the structure:

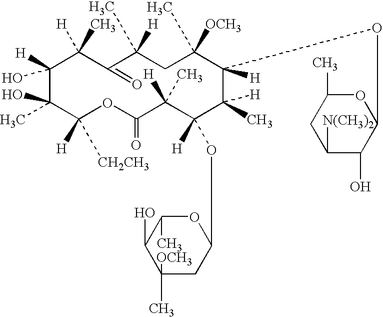

4. The injectable oil-in-water emulsion of claim 1 wherein the macrolide is at a concentration of about 2.5% by weight.

5. (Cancelled)

6. The injectable oil-in-water emulsion of claim 2 wherein the medium chain triglyceride is Miglyol 812, Crodamol GTCC-PN, or Neobees M-5 oil.

7. The injectable oil-in-water emulsion of claim 1 wherein the phospholipid is soy lecithin or egg lecithin.

8. The injectable oil-in-water emulsion of claim 1 further comprising a stabilizer.

9. The injectable oil-in-water emulsion of claim 8 wherein the stabilizer is glycine.

10. The injectable oil-in-water emulsion of claim 9 wherein the concentration of glycine is between about 0.1% and about 5% by weight.

11. The injectable oil-in-water emulsion of claim 8 wherein the stabilizer is ethylene diamine-tetraacetic acid (EDTA).

12. The injectable oil-in-water emulsion of claim 11 wherein the concentration of EDTA is between about 0.001% and about 0.01% by weight.

13. The injectable oil-in-water emulsion of claim 1 further comprising a tonicity modifier.

14. The injectable oil-in-water emulsion of claim 13 wherein the tonicity modifier is glycerol.

15. The injectable oil-in-water emulsion of claim 14 wherein the concentration of glycerol is between about 0.5% and about 2.5% by weight.

16. The injectable oil-in-water emulsion of claim 1 wherein the average size of the oil droplets in the emulsion is less than about 250 nm.

17. The injectable oil-in-water emulsion of claim 1 wherein the average size of the oil droplets in the emulsion does not increase more than 25% after storage at about 2-8° C. for 6 months.

18-23. (Cancelled).

24. An injectable oil-in-water emulsion, comprising:
(a) a macrolide at a therapeutically effective concentration,
(b) a vegetable oil,
(c) a phospholipid, and
(d) water,
wherein the emulsion does not cause vein irritation and is stable for at least 3 months.

25. The injectable oil-in-water emulsion of claim 24 further comprising a medium chain triglyceride.

26-48. (Cancelled).

49. A lyophilized formulation of a macrolide, wherein the formulation, when hydrated, produces the injectable oil-in-water emulsion according to claim 1.

50. The lyophilized formulation of claim 49 wherein the lyophilized formulation is reconstituted in a liquid medium to produce a reconstituted emulsion, and wherein the average droplet size of the reconstituted emulsion is no more than 200% of the average droplet size of the emulsion before lyophilization.
